(19) United States
(12) Patent Application Publication
(10) Pub. No.: US 2019/0383784 A1
(43) Pub. Date: Dec. 19, 2019

(54) PIPELINE INSPECTION ASSEMBLY USING SYNTHETIC WELD FLAWS

(71) Applicant: ExxonMobil Research and Engineering Company, Annandale, NJ (US)

(72) Inventors: Joseph W. Krynicki, The Woodlands, TX (US); Neeraj S. Thirumalai, Easton, PA (US); Lujian Peng, Humble, TX (US)

(21) Appl. No.: 16/430,832

(22) Filed: Jun. 4, 2019

Related U.S. Application Data
(60) Provisional application No. 62/684,876, filed on Jun. 14, 2018.

(51) Int. Cl. G01N 33/207

(52) U.S. Cl. CPC G01N 33/207

(57) ABSTRACT

A pipeline inspection assembly includes a test pipe including at least one weld and one or more synthetic weld flaws, and a pipeline inspection device mountable to the test pipe and movable relative thereto. The pipeline inspection device includes a detection device operable to detect the one or more synthetic weld flaws as the pipeline inspection device moves along a length of the test pipe. A performance of the pipeline inspection device is assessed based on a comparison of a detected characteristic of the one or more synthetic weld flaws and a known characteristic of the one or more synthetic weld flaws.

### CROSS REFERENCE TO RELATED APPLICATION

This application relates and claims priority to U.S. Provisional Application No. 62/684,876, filed on Jun. 14, 2018, entitled “Pipeline Inspection Assembly Using Synthetic Weld Flaws” which is incorporated herein in its entirety specifically by reference.

### BACKGROUND

Millions of miles of pipeline are used to carry a variety of fluids, such as gases, water, chemicals, and petroleum products (e.g., crude oil). Pipelines are vulnerable to internal and external corrosion, cracking, deformation, third party damage, and manufacturing flaws. In an attempt to keep pipelines operating safely, periodic inspections, such as In-Line Inspection (ILI) are performed to locate flaws and damage before they become cause for concern.

Some pipelines are made of ferrous materials that require longitudinal or lengthwise welds (e.g., “seam welds”) that help produce discrete pipe lengths from plates or strips, and further require circumferential welds (e.g., “girth welds”) used to connect adjacent pipes end-to-end. One common flaw in pipelines is associated with weld integrity or quality of seam and girth welds. During line pipe manufacturing as well as when a pipeline is built, trained inspection personnel use a variety of nondestructive testing (NDT) technologies and methods to evaluate pipeline welds and thereby ensure high quality. Beyond visual inspection, pipeline inspection devices have been developed and use technologies such as eddy-current, radiographic, ultrasonic, electromagnetic, and magnetic flux leakage inspection methods.

Prior to field service, pipeline inspection devices are commonly tested, calibrated, and/or qualified to assess NDT performance and probability of detection (POD). Ideally, naturally occurring weld flaws would be used to assess NDT performance, device calibration, and personnel training. However, natural weld flaws are impractical to harvest for extensive work and cannot be fully characterized without destructive testing. Instead, machined weld flaws (alternately referred to as “notches”) are commonly created and used during inspection demonstration work and for calibration and qualification of pipeline inspection tools. Machined flaws in seam and girth welds, however, do not accurately represent natural attributes of real (natural) weld flaws, such as curvature, angle changes, etc. These natural weld attributes can have a significant influence on the response of NDT technologies.

### SUMMARY

In accordance with the present disclosure, a pipeline inspection assembly is disclosed. The pipeline inspection assembly has a test pipe including at least one weld and one or more synthetic weld flaws and a pipeline inspection device mountable to the test pipe and movable relative thereto. The at least one weld is selected from the group consisting of a seam weld, a girth weld, and any combination thereof. The pipeline inspection device may include a detection device operable to detect the one or more synthetic weld flaws as the pipeline inspection device moves along a length of the test pipe. The performance of the pipeline inspection device is assessed based on a comparison of a detected characteristic of the one or more synthetic weld flaws and a known characteristic of the one or more synthetic weld flaws. The detection device uses a non-destructive testing technique selected from the group consisting of ultrasonic, electromagnetic, eddy-current, magnetic flux leakage, radiographic, and any combination thereof. The known characteristic of the one or more synthetic weld flaws is selected from the group consisting of size, curvature, angle, and any combination thereof.

In accordance with another aspect of the present disclosure, a method is disclosed. The method includes mounting a pipeline inspection device to a test pipe that includes at least one weld and one or more synthetic weld flaws. The pipeline inspection device includes a detection device. The method further includes advancing the pipeline inspection device along a length of the test pipe and detecting the one or more synthetic weld flaws with the detection device as the pipeline inspection device advances. Detecting the one or more synthetic weld flaws with the detection device includes using a non-destructive testing technique selected from the group consisting of ultrasonic, electromagnetic, eddy-current, magnetic flux leakage, radiographic, and any combination thereof. The further includes comparing a detected characteristic of the one or more synthetic weld flaws with a known characteristic of the one or more synthetic weld flaws and thereby assessing a performance of the pipeline inspection device.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a partial cross-sectional view of an example pipeline inspection assembly, according to one or more embodiments.

FIGS. 2A-2E are micrograph images of example weld flaws that may be simulated as synthetic weld flaws.

FIGS. 3A-3C are progressive views of the pipeline inspection assembly of FIG. 1 during example operation.

FIG. 4 is a graph 400 depicting a comparison between machined weld flaw detection and synthetic weld flaw detection.

FIG. 5 is a schematic flowchart of a method of assessing performance of a pipeline inspection device, according to one or more embodiments.

### DETAILED DESCRIPTION

The present disclosure is related to pipeline inspection techniques and, more particularly, to a pipeline inspection assembly that includes synthetic weld flaws that more accurately simulate naturally-occurring weld flaws and thereby enhance performance assessments of pipeline inspection devices.

Embodiments discussed herein describe a pipeline inspection assembly that includes a test pipe including at least one weld and one or more synthetic weld flaws, and a pipeline inspection device mountable to the test pipe and movable relative thereto. The pipeline inspection device includes a detection device operable to detect the one or more synthetic weld flaws as the pipeline inspection device moves along a length of the test pipe. A performance of the pipeline inspection device may be assessed based on a comparison of a detected characteristic of the one or more synthetic weld flaws and a known characteristic of the one or more synthetic weld flaws.

It can be critical to properly assess non-destructive testing (NDT) techniques of pipeline inspection devices, such as in-line-inspection (ILI) tools, with realistic weld flaws that simulate (mimic) real weld flaws in seam welds and/or girth welds of pipelines. According to embodiments of the present disclosure, synthetic weld flaws may be designed, fabricated, and used to assess and improve the performance of pipeline weld inspection. Traditional approaches are based on machined weld flaws, but the use of synthetic weld flaws allows a more accurate representation of naturally-occurring weld flaws. The proposed system and method improves the assessment of probability of detection (POD), sizing, and characterization of pipeline inspection devices.

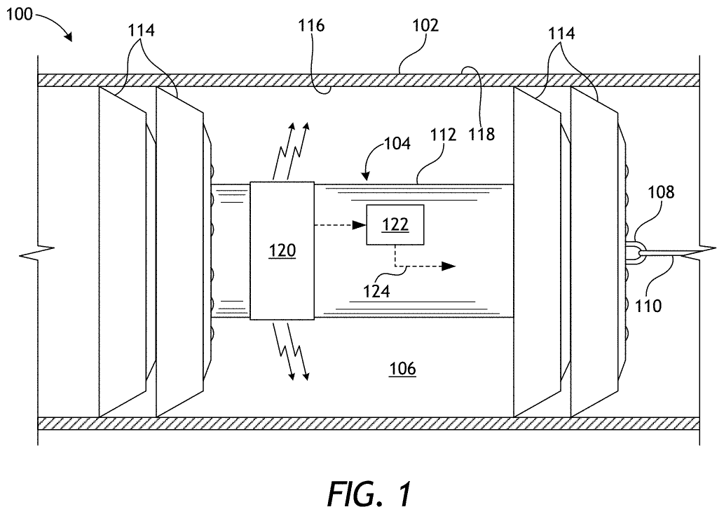

FIG. 1 is a partial cross-sectional view of an example pipeline inspection assembly 100, according to one or more embodiments. As illustrated the pipeline inspection assembly 100 (hereafter “the assembly 100”) includes a test pipe 102 and a pipeline inspection device 104 that may be mounted to the test pipe 102. In the illustrated embodiment, for example, the pipeline inspection device 104 (hereafter “the device 104”) is mounted to the test pipe 102 by being arranged within an interior 106 of the test pipe 102. In such embodiments, the device 104 may be characterized as an in-line inspection (ILI) device, alternatively referred to as a pipeline inspection gauge or “PIG.” In other embodiments, however, the device 104 may be mounted to the test pipe 102 by being arranged or otherwise used on the exterior of the test pipe 102, without departing from the scope of the disclosure. In such embodiments, the device 104 may comprise an automated tool or may otherwise comprise a manual inspection device that can be evaluated using the test pipe 102.

The test pipe 102, alternately referred to as a “test spool,” may be configured to simulate various characteristics of real pipelines that may be used in field service. Moreover, in some embodiments, the test pipe 102 may comprise a section of real pipeline, without departing from the scope of the disclosure. The device 104 may be configured to inspect the integrity of the test pipe 102 and thereby detect potential flaws and defects. Accordingly, the operational performance of the device 104 may be determined by advancing the device 104 along all or a portion of the test pipe 102 and reporting the detected characteristics.

As used herein, the term “pipeline” refers to any conduit in which a fluid can be moved (conveyed) and may alternately be referred to as a line pipe, piping, or a fluid conduit. In some embodiments, the test pipe 102 may simulate a pipeline used in the oil and gas industry to convey crude oil, a refinery product, an intermediate product, a chemical, or a gas. In such embodiments, the test pipe 102 may simulate (or comprise) any onshore or offshore flow system, such as mainline systems, long distance pipelines, risers, or flow lines used to transport untreated fluid between a wellhead and a processing facility, and flow lines used to transport hydrocarbon products, intermediate products, or byproducts as well as pipeline systems used to transport processed crude, products from refinery systems including gasoline, diesel, jet fuel, volatile liquids etc. In other embodiments, however, the test pipe 102 may simulate (or comprise) pipelines used in other industries, such as potable or sewer water pipelines, without departing from the scope of the disclosure.

In the illustrated embodiment, the device 104 may be configured to traverse the interior 106 of the test pipe 102 while simultaneously conducting non-destructive testing (NDT) to detect and size internal damage, such as weld flaws. In some embodiments, the device 104 may be a tethered device that can be pulled through the test pipe 102 or a section thereof. In such embodiments, the device 104 may include a coupling 108 arranged at a downstream end of the device 104 and a cable 110 may be attached to the coupling 108. Pulling on the cable 110 in the downstream direction (i.e., to the right in FIG. 1) may cause the device 104 to be advanced through the test pipe 102. In other embodiments, the device 104 may be non-tethered. In such embodiments, the device 104 may be self-propelled or may be pumped through the test pipe 102 from an upstream end.

In the illustrated embodiment, the device 104 may be capable of being pumped through the test pipe 102. More specifically, the device 104 may comprise a body 112 and one or more drive discs 114 (alternately referred to as piston seals, seal elements, or seal discs) coupled to or otherwise extending radially from the body 112. The drive discs 114 may be generally circular, having an outer circumference or periphery configured to engage or come into close engagement with an inner radial surface 116 of the test pipe 102. The drive discs 114 may be formed of polyurethane, but may alternatively be made of nylon, polyoxymethylene (POM, i.e., DELRIN™) polytetrafluoroethylene (PTFE, i.e., TEFLON™), an elastomer (e.g., rubber), or any combination thereof. The drive discs 114 may be flexible enough to create a fluid seal against the inner radial surface 116 of the test pipe 102, but compressible enough to help propel the device 104 through the test pipe 102 without excessive frictional resistance. While four drive discs 114 are depicted in the embodiment, the actual number of drive discs 114 may be more or less than four.

The assembly 100 may further include one or more detection devices 120 configured to monitor the test pipe 102 and detect flaws located on one or more of the inner radial surface 116, an outer radial surface 118 of the test pipe 102, and flaws located within the wall between the inner and outer radial surfaces 116, 118. The detection device 120 may employ one or more non-destructive testing (NDT) techniques to monitor the test pipe 102. Example NDT techniques or detection methods that may be employed by the detection device 120 include, but are not limited to, ultrasonic, electromagnetic, eddy-current, magnetic flux leakage, radiographic, or any combination thereof.

While only one detection device 120 is shown in FIG. 1, the assembly 100 may employ any number of detection devices 120, without departing from the scope of the disclosure. In some embodiments, for example, one or more detection devices 120 may be designed and otherwise capable of monitoring at generally every radial angle within the test pipe 102. The detection device(s) 120 may be able to continuously monitor seam welds and girth welds as the device 104 advances through the test pipe 102. The detection devices 120 may be strategically arranged at predetermined angular locations and thereby jointly monitor every radial angle of the test pipe 102. In such embodiments, the test pipe 102 may be divided into radial quadrants or other radial divisions and each radial quadrant or division may be monitored to allow the operator to map every radial angle of the inner and outer radial surfaces 116, 118 of the test pipe 102. This may prove advantageous in providing detection data and current pipeline 102 conditions found at each radial angle.

The detection device 120 may continuously or intermittently operate as the device 104 moves (advances) along the test pipe 102. Data obtained by the detection device 120 may be conveyed to a signal processor 122 communicably coupled to the detection device 120 and included in the assembly 100 or otherwise forming part thereof. The data obtained from the detection device 120 may be processed by or stored in the signal processor 122. For instance, the signal processor 122 may be a computer that includes a non-transitory machine-readable medium and configured to process the data and thereby provide an output signal 124 corresponding to one or more characteristics of the flaws detected. In other embodiments, the signal processor 122 may include an on-board memory or storage device configured to store the data received from the detection device 120. The stored data may be subsequently downloaded for processing resulting in the output signal 124.

The signal processor 122 may be communicably coupled to one or more communication interfaces (not shown) and otherwise configured to convey the output signal 124, either wired or wirelessly, to an external processing device (not shown) for consideration by an operator or for further processing and manipulation. One example communication interface may be a communication port (compatible with Ethernet, USB, etc.) defined or otherwise provided on the body 112 or any other portion of the device 104. The communication port may allow the signal processor 122 to be coupled to an external processing device, such as a computer, a hard drive, a handheld computer, a personal digital assistant (PDA), or other wireless transmission device. Once coupled thereto, the signal processor 122 may be able to download its stored data.

In other embodiments, the communication interface may be a wireless transmitter or link (not shown) arranged within the body 112. The signal processor 122 may be communicably coupled to the wireless link which may operate in accordance with any known wireless technology (e.g., Bluetooth, Wi-Fi, acoustic, etc.) and therefore be configured to wirelessly communicate with any remote wireless device such as, but not limited to, a radio, a cellular telephone, a PDA, a wireless network, satellite telecommunications, and the like. Accordingly, the signal processor 122 may be configured to wirelessly transmit the output signal 124 to the operator for consideration.

The foregoing description of the device 104 is merely one example of a pipeline inspection device that may be used in accordance with the principles of the present disclosure.

Consequently, the device 104 has been described merely for illustrative purposes and should not be considered limiting to the scope of the disclosure. In other embodiments, for example, instead of including the drive discs 114 (or in addition thereto), the device 104 may include a wheeled interface that engages the inner wall 116 of the pipeline 104. Moreover, as mentioned herein, the device 104 may alternatively be arranged external to the test pipe 102, without departing from the scope of the disclosure.

The device 104 may use one or more non-destructive testing (NDT) techniques to monitor and analyze the test pipe 102 for a variety of flaws that may be present at or near welded interfaces. More specifically, the test pipe 102 may include one or more longitudinal welds (i.e., “seam welds”) and/or one or more circumferential welds (i.e., “girth welds”). The seam and girth welds may be produced through electrical resistance welding, but could alternatively be produced through other types of welding technologies.

Prior to deploying the device 104 for field use, the device 104 may be tested against the test pipe 102 to assess its performance and determine its probability of detection (POD). This may be accomplished by allowing the device 104 to monitor and detect characteristics of known weld flaws included on (or in) the test pipe 102. More specifically, one or more known weld flaws may be provided at known locations within the test pipe 102 and may exhibit known characteristics, such as size (e.g., length, depth, height, etc.), curvature, and angle. The device 104 may be advanced past the known weld flaws and the output signal 124 may be analyzed to determine if the device 104 is capable of accurately detecting the known weld flaws. Based on the accuracy of the device 104, the POD of the device 104 may be determined. The ability of the device 104 to size and characterize the weld flaws may also be determined.

The known weld flaws are intended to mimic or substantially mimic real or naturally occurring weld flaws. Real or naturally occurring weld flaws are weld flaws that develop in a component during its manufacture or service without any steps having been taken to deliberately encourage its development. Service-related weld flaws develop in service with stress and time and are generally not created by the welding process. However, service flaws can also develop from the growth of non-injurious (e.g., very small) weld flaws. Examples of real weld flaws include, but are not limited to, incomplete fusion, incomplete penetration, incomplete sidewall or inter-run fusion, joint misalignment, porosity, solidification cracking, cluster cracking, heat affected zone (hydrogen) cracking, undercut, brittle fracture or fatigue cracks, under or overfill of weld metal, hook cracks, cold welds, excessive trim, inadequate flash trim, and inclusions (slag or metallic).

Naturally occurring weld flaws would be ideal for assessing the performance of the device 104 and for training purposes, but they are impractical to harvest for extensive work and they cannot be fully characterized without destruction. Accordingly, known weld flaws are often generated by machining flaws into the test pipe at known locations. A machined weld flaw can comprise a cut or a machined void produced in or immediately adjacent a welded interface, such as a seam or girth weld. Machined weld flaws can be generated through saw cuts, machined holes, machined slots, collectively referred to herein as “machined notches.” Electrical discharge machining (EDM) is perhaps the most relied upon technology to generate machined notches, but other mechanical devices can also be used to make the machined notches.

Machined weld flaws, however, may not accurately represent the natural attributes (i.e., geometry) of real weld flaws, such as curvature and angle changes. As will be appreciated by those skilled in the art, such attributes can have a significant influence on the response of non-destructive inspection technologies. It, however, is contemplated that machined notches may be used in connection with the presently disclosed subject matter. The machined notches in connection with the synthetic flaws may be used to assist with assessing the accuracy of NDT techniques including but not limit to ILI tools.

According to embodiments of the present disclosure, the test pipe 102 may include one or more synthetic weld flaws that better mimic (simulate) naturally occurring weld flaws, and thereby provide a more accurate performance determination of the device 104. More particularly, using synthetic weld flaws as opposed to machined weld flaws enables a superior assessment of the POD of the device 104 (or any other pipe inspection device) using a non-destructive testing technology, and its ability to size and characterize real weld flaws. Synthetic weld flaws (alternately referred to as “artificial realistic flaws”) may comprise weld flaws that are generated without machining and produce a response to non-destructive testing methods under assessment that resembles that of a real weld flaw, including service-related flaws. Synthetic weld flaws can include, but are not limited to, incomplete side-wall fusion, incomplete fusion, slag inclusion, solidification cracking, a cluster crack, weld metal transverse cracking, porosity, heat affected zone (HAZ) cracking, and brittle fracture and fatigue cracks.

Synthetic weld flaws may be generated in a variety of ways. In some embodiments, for example, a synthetic weld flaw may comprise a pre-existing real weld flaw that is mechanically attached to a surface (i.e., inner or outer surface 116, 118) of the test pipe 102. In other embodiments, a synthetic weld flaw may be generated through weld doping or weld modification where, for instance, a crack prone material is added to a weld to promote localized weld cracking. Porosity or slag may similarly be introduced to generate the synthetic weld flaw. In yet other embodiments, the synthetic weld flaw may be “grown,” where cracking may be initiated and propagated into the test pipe 102 via processes such as thermal fatigue and stress corrosion cracking.

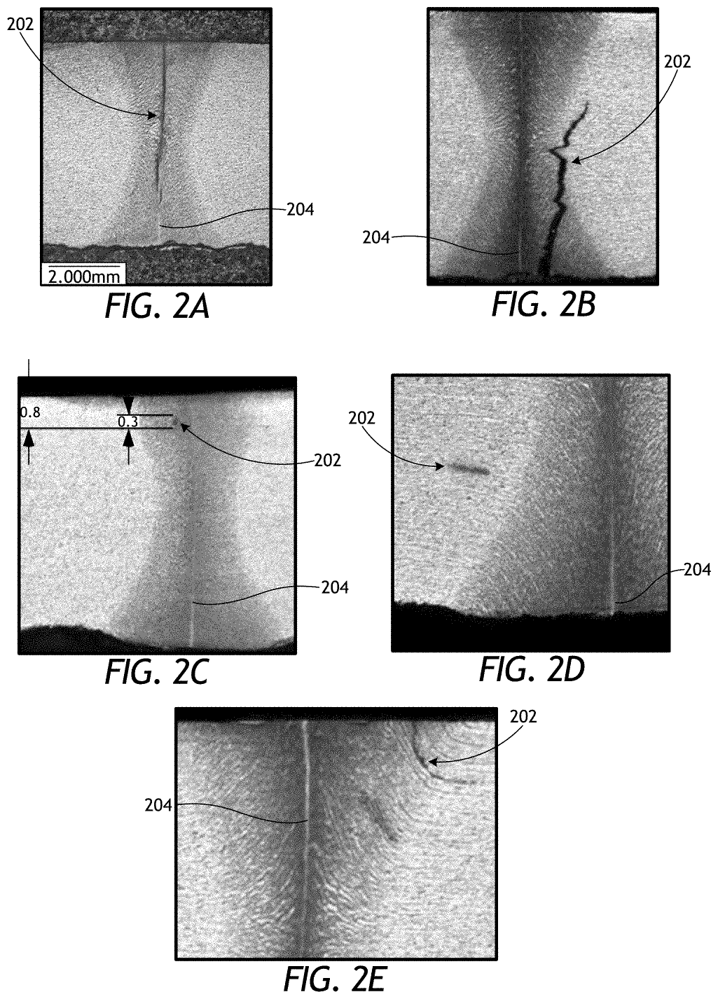

FIGS. 2A-2E are micrograph images of example weld flaws 202 that may be replicated (simulated) as synthetic weld flaws, in accordance with one or more embodiments of the present disclosure. More particularly, FIGS. 2A-2E depict common fabrication or service-related weld flaws 202 located at or immediately adjacent a weld interface 204. The weld interface 204 may form part of a seam weld or a girth weld, for example, but may alternatively form part of any other welded interface that might be present in a pipeline.

In FIG. 2A, the weld flaw 202 comprises a cold weld flaw, which is a substantially planar flaw and extends generally parallel with the weld interface 204. In FIG. 2B, the weld flaw 202 comprises a hook crack that extends at an angle offset from the weld interface 204. Notably, the hook crack is complex in terms of its jaggedness. In FIG. 2C, the weld flaw 202 comprises a buried inclusion, which may comprise a non-metallic or intermetallic inclusion. In FIG. 2D, the weld flaw 202 comprises a lamination, which is a flaw that extends generally parallel to the inner and outer surfaces of the test pipe 102 (FIG. 1). In FIG. 2E, the weld flaw 202 comprises an upturned fiber, which comprises separation in the material that can ultimately develop into a hook crack.

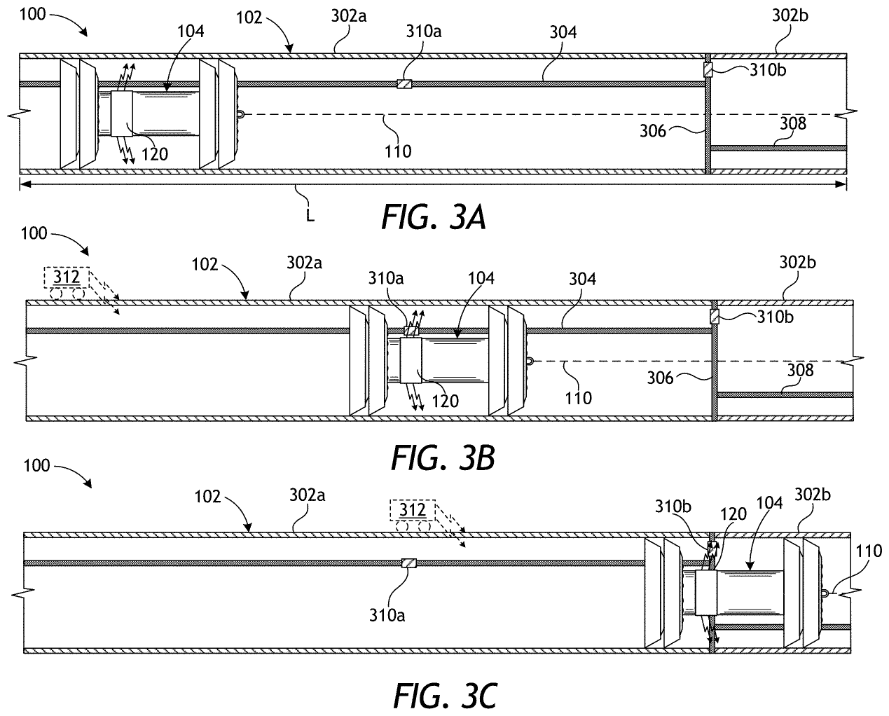

FIGS. 3A-3C are progressive views of the assembly 100 during example operation in assessing performance of the device 104, according to one or more embodiments. In the illustrated embodiment, the test pipe 102 includes a first pipe section 302a and a second pipe section 302b coupled to the first pipe section 302a in an end-to-end fashion. In other embodiments, however, the test pipe 102 may include only a single length of pipe, without departing from the scope of the disclosure.

As shown in FIG. 3A, the test pipe 102 may exhibit a length L, and the device 104 may be configured to be advanced along all or a portion of the length L during testing. The length L may vary depending on the application. In some embodiments, for example, the length L may range between about 10 feet and about 20 feet, but may alternatively be less than 10 feet or greater than 20 feet (or any distance therebetween), without departing from the scope of the disclosure.

The test pipe 102 includes one or more welds (or welded interfaces), shown as a seam weld 304 and a girth weld 306. The seam weld 304 extends longitudinally along the first pipe section 310a. In some embodiments, the second pipe section 310b may also include a seam weld 308. The girth weld 306 may help couple the first and second pipe sections 310a,b.

In the illustrated embodiment, the test pipe 102 may include one or more synthetic weld flaws, shown as a first synthetic weld flaw 310a and a second synthetic weld flaw 310b. The synthetic weld flaws 310a,b may comprise any of the synthetic weld flaws mentioned or described herein. Consequently, and for simplicity, the synthetic weld flaws 310a,b are depicted generally as a box and not specific to any particular synthetic weld flaw. As illustrated, the first synthetic weld flaw 310a may be located at or immediately adjacent the seam weld 304 at a first known location along the length L, and the second synthetic weld flaw 310b may be located at or immediately adjacent the girth weld 306 at a second known location along the length L. While only two synthetic weld flaws 310a,b are depicted in FIGS. 3A-3C, more or less than two may be included in the test pipe 102, without departing from the scope of the disclosure.

In some embodiments, the first and second synthetic weld flaws 310a,b may simulate (i.e., be representative of) naturally-occurring weld flaws expected to be encountered in a certain pipeline in field use. More specifically, some naturally-occurring weld flaws may be more evident or likely in pipelines located in a particular geographic region and pipelines that convey particular fluids. Accordingly, the test pipe 102 may be designed such that the synthetic weld flaws 310a,b simulate real weld flaws that are likely to develop in the particular pipeline scenario. In other embodiments, or in addition thereto, the synthetic weld flaws 310a,b may be designed based on the device 104. More particularly, the synthetic weld flaws 310a,b may comprise weld flaw types that the device 104 may traditionally have difficulty detecting. This may prove advantageous in identifying weaknesses/limitations of the device 104 and anticipating performance issues.

In example operation, the device 104 may be advanced in a first direction (e.g., downstream or to the right in FIGS. 3A-3C) within the test pipe 102. The device 104 may be pulled or pushed along the length L of the test pipe 102. For example, the device 104 may be pulled by pulling on the cable 110 (shown in dashed lines) in the first direction. Alternatively, the device 104 may be pushed (impelled) by being pumped through the test pipe 102 from an upstream end of the test pipe 102. In yet other embodiments, the device 104 may be self-propelled, such as including wheels (not shown) and a drive transmission that rotates the wheels to advance the device 104.

As the device 104 advances, the detection device 120 may continuously or intermittently operate to monitor the integrity of the welds 304, 306. In FIG. 3B, the device 104 has encountered the first synthetic weld flaw 310a and the detection device 120 may correspondingly obtain data relating to the characteristics of the first synthetic weld flaw 310a. In FIG. 3C, the device 104 has encountered the second synthetic weld flaw 310b and the detection device 120 may correspondingly obtain data relating to the characteristics of the second synthetic weld flaw 310b.

The characteristic data obtained by the device 104 may then be compared against the true and known characteristics of the first and second weld flaws 310a,b to assess the performance (e.g., sizing and characterization) and probability of detection (POD) of the device 104. This comparison provides the operator with an indication of whether the device 104 actually detected each synthetic weld flaw 310a,b, and determines how well the device 104 sizes the synthetic weld flaws 310a,b; e.g., the depth, length, height, angle, etc. The comparison may also show whether the device 104 accurately characterized the synthetic weld flaws 310a,b; e.g., whether the synthetic weld flaws 310a,b comprise a crack, a gouge, a lamination, an inclusion, etc.

In some embodiments, the comparison between the characteristic data obtained by the device 104 and the true and known characteristics of the synthetic weld flaws 310a,b may be used to help train or calibrate the device 104. This may be accomplished by understanding and evaluating the NDT signal response from specific features and subsequently developing improved hardware, software, or interpretation guidelines to improve performance. For example, the timing and amplitude of an ultrasonic signal can be different based on flaw tilt, and algorithms may be developed to improve detection, sizing, and characterization capabilities.

Moreover, using synthetic weld flaws may also prove advantageous in improving training for field NDT technicians. More specifically, synthetic weld flaws are more realistic and closer to real weld flaws as opposed to machined weld flaws. Consequently, the ability of the field NDT technicians to detect real weld flaws may be assessed based on the synthetic weld flaws, and more effective training may be provided as a result. Field NDT technicians have limited opportunities to test and train on naturally occurring flaws. Therefore, the use of synthetic weld flaws provides the opportunity for more advanced training, which is representative of real flaws (which are inherently more difficult to detect/size).

While the device 104 is depicted in FIGS. 3A-3C as traversing the interior of the test pipe 102, it is also contemplated herein to employ and assess an externally mounted pipe inspection device. Referring specifically to FIGS. 3B and 3C, for instance, a second pipeline inspection device 312 (shown in dashed lines) is depicted mounted to the exterior of the test pipe 102. The second pipeline inspection device 312 (hereafter “the second device 312”) may be substantially similar to the device 104 in that it may include one or more detection devices (not shown) capable of monitoring the test pipe 102 and detecting flaws located on the inner and outer radial surfaces thereof using one or more non-destructive testing (NDT) techniques. In FIG. 3C, the second device 312 is shown advanced along the test pipe 102. As will be appreciated, the second device 312 may be advanced by pushing or pulling the second device 312 in the desired direction, but the second device 312 may alternatively be self-propelled.

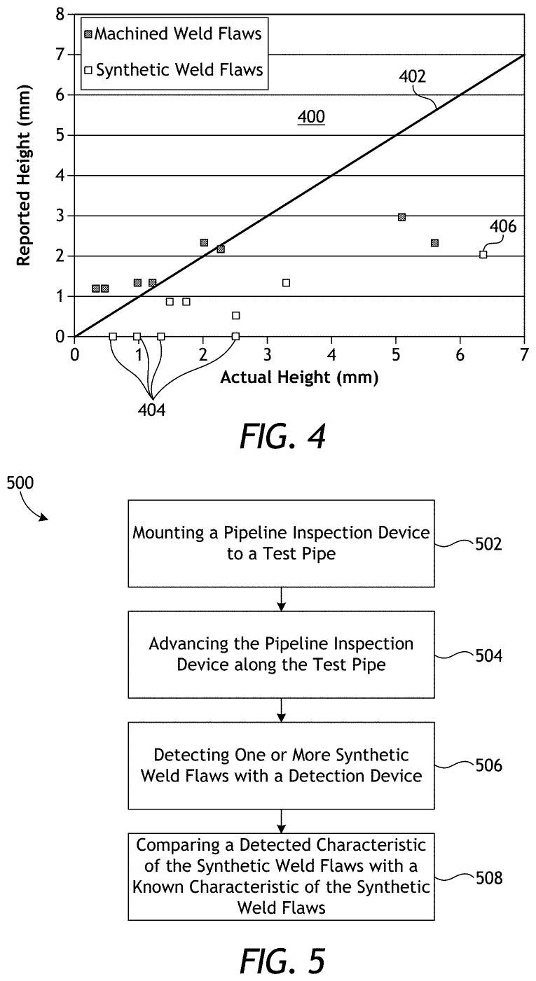

FIG. 4 is a graph 400 depicting a comparison between machined weld flaw detection and synthetic weld flaw detection. More specifically, the graph 400 depicts data results obtained from a pipeline inspection device (e.g., the device 104 of FIGS. 1 and 3A-3C) upon detecting a plurality of machined weld flaws and a plurality of synthetic weld flaws in a test pipe (e.g., the test pipe 102 of FIGS. 1 and 3A-3C). As compared to machined weld flaws, synthetic weld flaws are more difficult to detect and size, which is consistent with field experience in detecting naturally-occurring weld flaws. If the pipeline inspection device were accurate, most of the results would be plotted at or near the parity line 402.

As illustrated, each of the machined weld flaws were detected by the pipeline inspection device, but four synthetic weld flaws 404 were entirely undetected by the pipeline inspection device. Moreover, the actual height of one synthetic weld flaw 406 was about 6.3 mm, but the pipeline inspection device reported the height of the synthetic weld flaw 406 to be about 2 mm, which is a significant difference. Accordingly, the graph 400 demonstrates that the pipeline inspection device was capable of detecting machined weld flaws, but had poor performance in detecting synthetic weld flaws, which are more analogous to real weld flaws.

FIG. 5 is a schematic flowchart of a method 500 of assessing performance of a pipeline inspection device, according to one or more embodiments. As illustrated, the method may include mounting a pipeline inspection device to a test pipe, as at 502. The test pipe may include at least one weld and one or more synthetic weld flaws, and the pipeline inspection device may include a detection device. The pipeline inspection device may then be advanced along a length of the test pipe, as at 504. As the pipeline inspection device advances, the one or more synthetic weld flaws may be detected with the detection device, as at 506. The method 500 may then include comparing a detected characteristic of the one or more synthetic weld flaws with a known characteristic of the one or more synthetic weld flaws, as at 508. Based on this comparison, a performance and probability of detection of the pipeline inspection device may be assessed.

It is contemplated herein that the method 500 may be undertaken or otherwise carried out at any desired location to assess the performance of a pipeline inspection device. In some embodiments, for example, the method 500 may be undertaken at a testing facility, alternately referred to as a “shop.” The testing facility may be a controlled environment that includes one or more test pipes that may be used to assess the capability of a pipeline inspection device. In other embodiments, the method may be undertaken at a field location. As used herein, the term “field location” refers to an on-site geographical location where an in-use pipeline is erected for use. In such embodiments, a test pipe may be provided at the field location to not only assess the performance (capability) of a pipeline inspection device but to also verify that the pipeline inspection device is calibrated and operates properly while inspecting the in-use pipeline.

It is also contemplated herein that the principles of the present disclosure may be used for training purposes. More specifically, the method 500 may be undertaken to help train inspection personnel, such as an ultrasonic field technician. In such embodiments, the inspection personnel may improve their proficiency related to identifying and characterizing seam crack flaws. The method 500 may alternatively, or in addition thereto, be used to improve (train) ILI interpretation algorithms programmed to a particular pipeline inspection device. In yet other embodiments, the method 500 may be used to improve (train) ILI data analysts. Training of technicians and analysts may be accomplished by enabling them to compare nondestructive evaluation (NDE) signal responses to synthetic flaws (which have known characteristics, e.g., flaw type, dimension, etc.).

### EMBODIMENTS DISCLOSED HEREIN INCLUDE

A. A pipeline inspection assembly includes a test pipe including at least one weld and one or more synthetic weld flaws, and a pipeline inspection device mountable to the test pipe and movable relative thereto, the pipeline inspection device including a detection device operable to detect the one or more synthetic weld flaws as the pipeline inspection device moves along a length of the test pipe, wherein a performance of the pipeline inspection device is assessed based on a comparison of a detected characteristic of the one or more synthetic weld flaws and a known characteristic of the one or more synthetic weld flaws.

B. A method includes mounting a pipeline inspection device to a test pipe that includes at least one weld and one or more synthetic weld flaws, wherein the pipeline inspection device includes a detection device, advancing the pipeline inspection device along a length of the test pipe, detecting the one or more synthetic weld flaws with the detection device as the pipeline inspection device advances, and comparing a detected characteristic of the one or more synthetic weld flaws with a known characteristic of the one or more synthetic weld flaws and thereby assessing a performance of the pipeline inspection device.

Each of embodiments A and B may have one or more of the following additional elements in any combination: Element 1: wherein the at least one weld is selected from the group consisting of a seam weld, a girth weld, and any combination thereof. Element 2: wherein the detection device uses a non-destructive testing technique selected from the group consisting of ultrasonic, electromagnetic, eddy-current, magnetic flux leakage, radiographic, and any combination thereof. Element 3: wherein the pipeline inspection device is mounted within an interior of the test pipe. Element 4: wherein the pipeline inspection device is mounted to an exterior of the test pipe. Element 5: wherein the pipeline inspection device comprises a tethered device that is pulled through an interior of the test pipe. Element 6: wherein the pipeline inspection device is pumped through an interior of the test pipe. Element 7: wherein the pipeline inspection device is self-propelled. Element 8: wherein the performance of the pipeline inspection device comprises a probability of detection. Element 9: wherein the known characteristic of the one or more synthetic weld flaws is selected from the group consisting of size, curvature, angle, and any combination thereof. Element 10: wherein the one or more synthetic weld flaws simulate naturally occurring weld flaws including both manufacture and service related flaws. Element 11: wherein detecting the one or more synthetic weld flaws with the detection device comprises using a non-destructive testing technique selected from the group consisting of ultrasonic, electromagnetic, eddy-current, magnetic flux leakage, radiographic, and any combination thereof. Element 12: wherein mounting the pipeline inspection device to the test pipe comprises arranging the pipeline inspection device within an interior of the test pipe. Element 13: wherein mounting the pipeline inspection device to the test pipe comprises mounting the pipeline inspection device to an exterior of the test pipe. Element 14: wherein advancing the pipeline inspection device comprises pulling the pipeline inspection device through an interior of the test pipe. Element 15: wherein advancing the pipeline inspection device comprises pumping the pipeline inspection device through an interior of the test pipe. Element 16: further comprising training field nondestructive testing technicians based on the one or more synthetic weld flaws. Element 17: further comprising calibrating the pipeline inspection device based on the one or more synthetic weld flaws. Element 18: wherein the test pipe is located at a testing facility. Element 19: wherein the test pipe is located at a field location. Element 20: further comprising training at least one of an in-line inspection (ILI) algorithm and an ILI data analyst based on the one or more synthetic weld flaws.

By way of non-limiting example, exemplary combinations applicable to A and B include: Element 1 with Element 3; Element 1 with Element 4; Element 3 with Element 5; Element 3 with Element 6; Element 3 with Element 7; Element 4 with Element 7; Element 9 with Element 10; Element 12 with Element 14; Element 12 with Element 15; Element 11 with Element 17; Element 12 with Element 18; Element 12 with Element 19; and Element 16 with Element 20.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces.

If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

1. A pipeline inspection assembly, comprising:
a test pipe including at least one weld and one or more synthetic weld flaws; and
a pipeline inspection device mountable to the test pipe and movable relative thereto, the pipeline inspection device including a detection device operable to detect the one or more synthetic weld flaws as the pipeline inspection device moves along a length of the test pipe,
wherein a performance of the pipeline inspection device is assessed based on a comparison of a detected characteristic of the one or more synthetic weld flaws and a known characteristic of the one or more synthetic weld flaws.

2. The pipeline inspection assembly of claim 1, wherein the at least one weld is selected from the group consisting of a seam weld, a girth weld, and any combination thereof.

3. The pipeline inspection assembly of claim 1, wherein the detection device uses a non-destructive testing technique selected from the group consisting of ultrasonic, electromagnetic, eddy-current, magnetic flux leakage, radiographic, and any combination thereof.

4. The pipeline inspection assembly of claim 1, wherein the pipeline inspection device is mounted within an interior of the test pipe.

5. The pipeline inspection assembly of claim 1, wherein the pipeline inspection device is mounted to an exterior of the test pipe.

6. The pipeline inspection assembly of claim 1, wherein the pipeline inspection device comprises a tethered device that is pulled through an interior of the test pipe.

7. The pipeline inspection assembly of claim 1, wherein the pipeline inspection device is pumped through an interior of the test pipe.

8. The pipeline inspection assembly of claim 1, wherein the pipeline inspection device is self-propelled.

9. The pipeline inspection assembly of claim 1, wherein the performance of the pipeline inspection device comprises a probability of detection.

10. The pipeline inspection assembly of claim 1, wherein the known characteristic of the one or more synthetic weld flaws is selected from the group consisting of size, curvature, angle, and any combination thereof.

11. The pipeline inspection assembly of claim 1, wherein the one or more synthetic weld flaws simulate naturally occurring weld flaws including both manufacture and service related flaws.

12. A method, comprising:
mounting a pipeline inspection device to a test pipe that includes at least one weld and one or more synthetic weld flaws, wherein the pipeline inspection device includes a detection device;
advancing the pipeline inspection device along a length of the test pipe;
detecting the one or more synthetic weld flaws with the detection device as the pipeline inspection device advances; and
comparing a detected characteristic of the one or more synthetic weld flaws with a known characteristic of the one or more synthetic weld flaws and thereby assessing a performance of the pipeline inspection device.

13. The method of claim 12, wherein detecting the one or more synthetic weld flaws with the detection device comprises using a non-destructive testing technique selected from the group consisting of ultrasonic, electromagnetic, eddy-current, magnetic flux leakage, radiographic, and any combination thereof.

14. The method of claim 12, wherein mounting the pipeline inspection device to the test pipe comprises arranging the pipeline inspection device within an interior of the test pipe.

15. The method of claim 12, wherein mounting the pipeline inspection device to the test pipe comprises mounting the pipeline inspection device to an exterior of the test pipe.

16. The method of claim 12, wherein advancing the pipeline inspection device comprises pulling the pipeline inspection device through an interior of the test pipe.

17. The method of claim 12, wherein advancing the pipeline inspection device comprises pumping the pipeline inspection device through an interior of the test pipe.

18. The method of claim 12, further comprising training field nondestructive testing technicians based on the one or more synthetic weld flaws.

19. The method of claim 12, further comprising calibrating the pipeline inspection device based on the one or more synthetic weld flaws.

20. The method of claim 12, wherein the test pipe is located at a testing facility.

21. The method of claim 12, wherein the test pipe is located at a field location.

22. The method of claim 12, further comprising training at least one of an in-line inspection (ILI) algorithm and an ILI data analyst based on the one or more synthetic weld flaws.
